(19) United States
(12) Patent Application Publication
(10) Pub. No.: US 2019/0001080 A1
(43) Pub. Date: Jan. 3, 2019

(54) ACCESSORY DEVICE FOR METERED DOSE INHALER AND METHODS OF USE

(71) Applicant: Cyril OKADIGWE, Taylors, SC (US)

(72) Inventor: Cyril OKADIGWE, Taylors, SC (US)

(21) Appl. No.: 15/639,133

(22) Filed: Jun. 30, 2017

(51) Int. Cl. A61M 15/00; A61K 9/00

(52) U.S. Cl. CPC A61M 15/0001; A61M 15/0068; A61M 15/0021; A61M 15/009; A61K 9/008

(57) ABSTRACT

An accessory device for use with a hand-held metered dose inhaler (MDI), comprising: a housing, sized and shaped to mount the MDI therein; a movement mechanism configured to cause compressive movement of at least one piece of the housing to activate the MDI; and, at least one side-mounted movement actuator operationally connected to the movement mechanism to instigate the movement.

### FIELD AND BACKGROUND OF THE INVENTION

The present invention, in some embodiments thereof, relates to medical 15 devices and, more particularly, but not exclusively, to making medical devices easier to use, improving patient compliance and/or enhancing patient medical outcomes.

Hand held metered dose inhalers (“MDI”) are meant to be actuated between the thumb and index fingers. However, MDI can be very difficult to use by patients with degenerative diseases, such as osteoarthritis or rheumatoid arthritis, or by patients 20 who have suffered hand injuries, or by patients who otherwise lack sufficient manual control for operation of the MDI, such as young children.

Previous attempts have been made to increase the usability of MDI, including: U.S. Pat. No. 8,695,589 to Mullane, et al., which describes an inhaler assist device shaped and dimensioned for supporting a metered dose inhaler and an associated anti-static 25 chamber in a manner allowing for assisted compression of the metered dose inhaler to dispense a dosage therefrom.

U.S. Pat. No. 6,681,763 and U.S. Pat. No. 6,397,837 to Ferris, describe methods and apparatuses for enabling a user having reduced manual dexterity and/or strength to operate an aerosol inhaler by modifying the inhaler to have a lever juxtaposed adjacent the flat end of the 30 aerosol canister, or inserting an inhaler into a device having a lever juxtaposed to the flat end of the aerosol canister, the lever adapted to be moved by the user to depress the aerosol canister and dispense medication contained in said aerosol canister.

U.S. Pat. No. 5,133,343 to Johnson, I V, et al., describes an apparatus, having an outlet port insertable within a user's mouth, having housed therein an automatically actuated 35 commercially available and replaceable inhaler for discharging a medicated vapor through the outlet port into the user's mouth upon inhalation.

U.S. D683,443 to Mullane, et al., describes the ornamental features of an inhaler assist device.

### SUMMARY OF THE INVENTION

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example, are not necessarily to scale, and are for purposes of illustrative discussion of embodiments of the invention. In this regard, the description taken with the drawings makes apparent to those skilled in the art how embodiments of the invention may be practiced.

In the drawings:

FIG. 1 is a perspective view of a hand held metered dose inhaler;

FIG. 2 is a block diagram of an accessory device for a hand held metered dose inhaler;

FIG. 3 is a perspective view of a hand held metered dose inhaler with an exemplary accessory device attached thereto;

FIG. 4 is a perspective view of the accessory (without a hand held metered dose inhaler);

FIGS. 5A-5F are front, back, left, right, top and bottom views, respectively, of the accessory of FIG. 4;

FIG. 6 is an exploded view of the accessory of FIG. 4; and,

FIG. 7 is a flowchart showing a method of using an accessory device for a hand held metered dose inhaler.

### DESCRIPTION OF SPECIFIC EMBODIMENTS OF THE INVENTION

The present invention, in some embodiments thereof, relates to medical devices and, more particularly, but not exclusively, to making medical devices easier to use, improving patient compliance and/or enhancing patient medical outcomes.

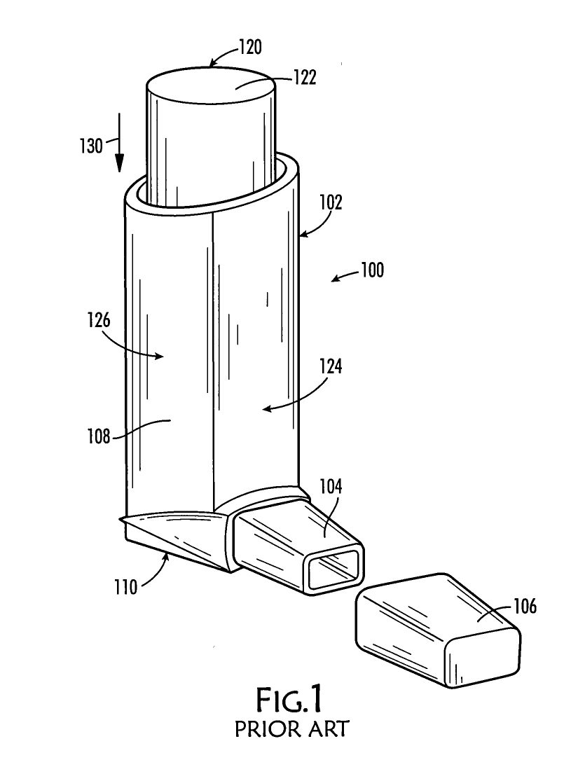

For purposes of better understanding some embodiments of the present invention, reference is first made to the construction and operation of a hand held MDI 100, as illustrated in FIG. 1. FIG. 1 is a perspective view of a MDI 100, consisting of housing 102, with a mouthpiece 104, the mouthpiece 104 adapted to be closed by a mating cover 106. Housing 102 has a body portion 108 which receives an aerosol container 120, the aerosol canister 120 having a first end (not shown) and a second or generally flat (bottom) end 122. The first end of the aerosol canister, can or receptacle 120 is well known and one type is shown for example in U.S. Pat. No. 3,456,644, the description of which is incorporated herein by reference. Activation of the MDI 100 is performed by pressing on the end 122 of the aerosol canister 120 downward (in a vertical axis 130 direction) with at least one finger while holding the bottom 110 of the MDI 100 with another finger, usually the thumb. Generally, MDIs are 4-sided and have a rectangular or square cross-section. As used herein, “side” refers to the two sides 124 of the MDI (and/or the two sides of the accessory device 200 corresponding to those two sides of the MDI) which are not the part of the MDI with the mouthpiece 104 (referred to as the “front” 126) or the part opposite the front (referred to as the “back”, but not shown in FIG. 1 because it is a perspective view).

Before explaining at least one embodiment of the invention in detail, it is to be understood that the invention is not necessarily limited in its application to the details of construction and the arrangement of the components and/or methods set forth in the following description and/or illustrated in the drawings. The invention is capable of other embodiments or of being practiced or carried out in various ways.

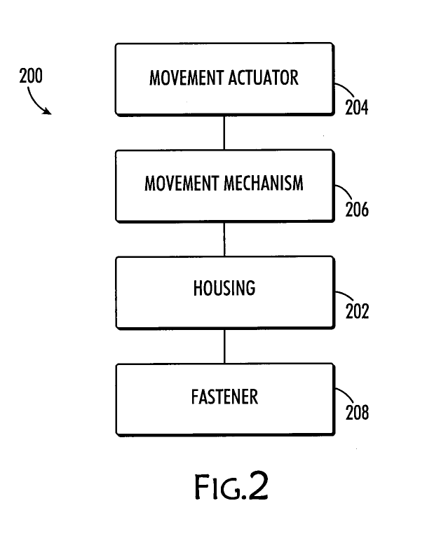

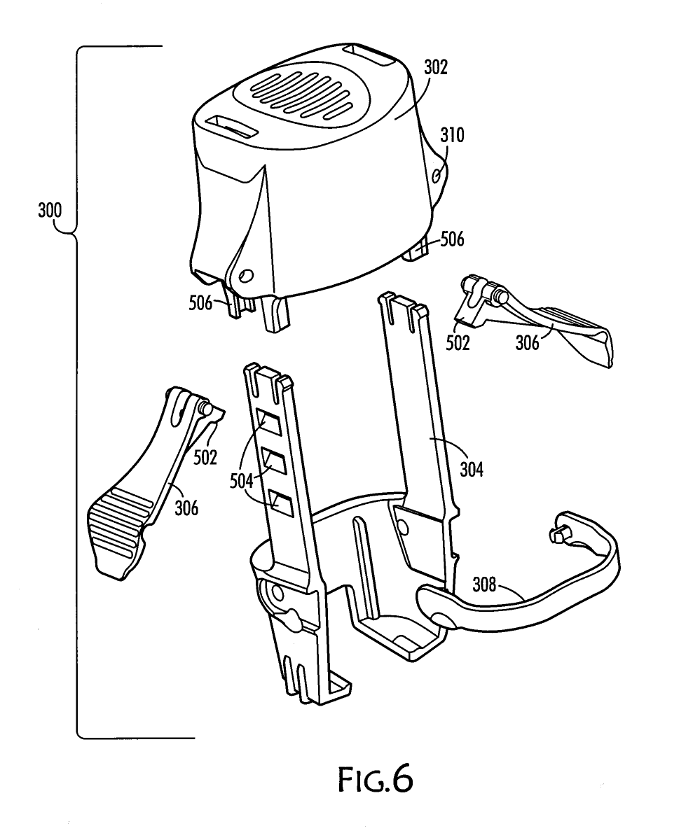

Referring now to the drawings, FIG. 2 is a block diagram of an accessory device 200 for a hand held MDI 100. In some aspects, the accessory device 200 includes a housing for removable placement/mounting of a MDI 100 therein. Optionally, the housing 202 is comprised of a plurality of pieces, such as shown and described with respect to FIG. 6. At least one side-mounted movement actuator 204 is provided to the accessory device 200 to instigate movement of a movement mechanism 206, which is configured to cause the activation of the MDI 100. It should be understood that “side-mounted” as used herein means that an actuator is attached to the housing on at least one of the two sides and that movement is initiated, and the MDI is activated, by transferring the conventional vertical axis 130 of motion for activation to a horizontal axis of motion (i.e. transverse to axis 130).

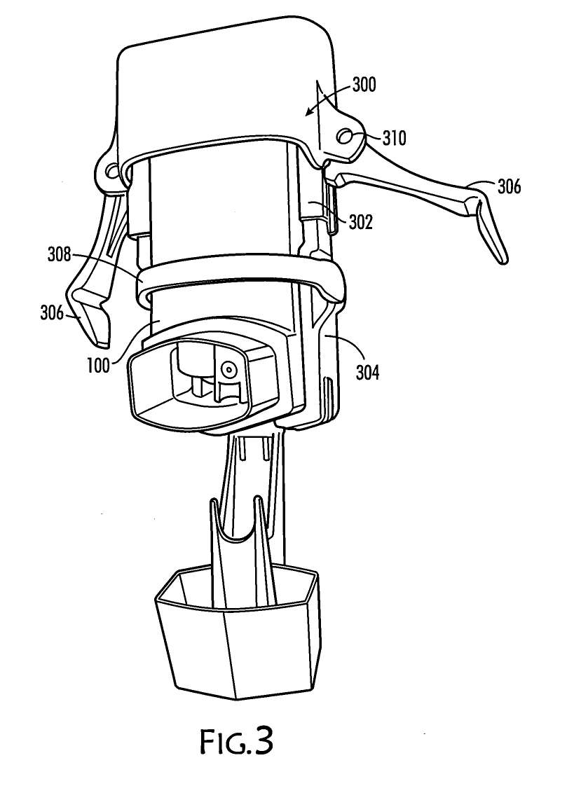

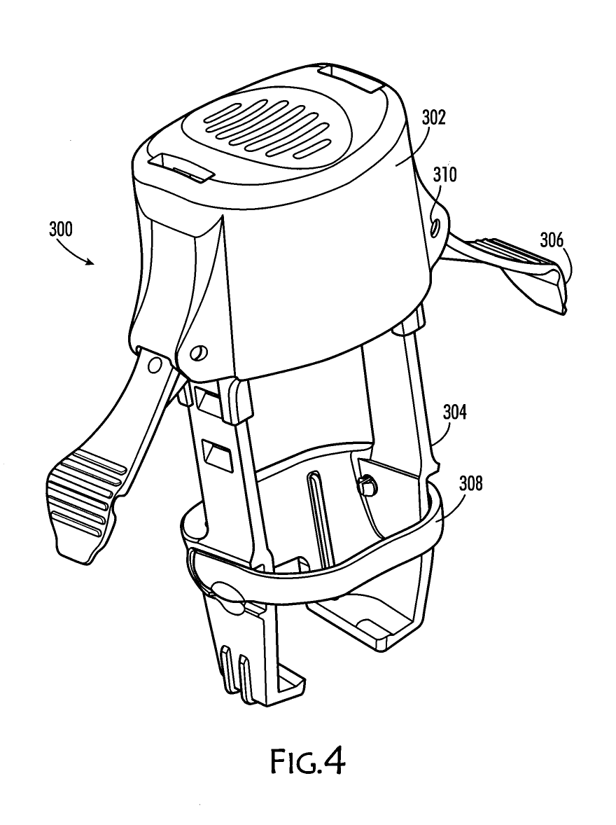

In an aspect, the at least one movement actuator 204 is a lever or levers, for example as shown and described with respect to FIGS. 3-4, inter alia. In an aspect, the movement mechanism 206 is one which enables compressive movement on the MDI, which in turn causes activation of the MDI (which are conventionally designed for activation by vertically compressing the aerosol canister 120 in a direction towards the mouthpiece). Optionally, the accessory device 200 includes a removably attachable fastener 208 of some sort to at least partially enclose the MDI to retain the MDI within the housing 202. Exemplary fasteners 208 include a strap or bar, a resilient element like an elastic band, a snap-in interface, and the like.

FIG. 3 is a perspective view of a hand held MDI 100 with an exemplary accessory device 300 attached thereto, in an aspect. It should be understood that accessory device 300 is but one example of carrying-out an accessory device 200 for making MDIs easier to use, improving patient compliance and/or enhancing patient medical outcomes by improving patient dosing using MDIs. Other possible embodiments generally conforming to accessory device 200 are conceived by this disclosure.

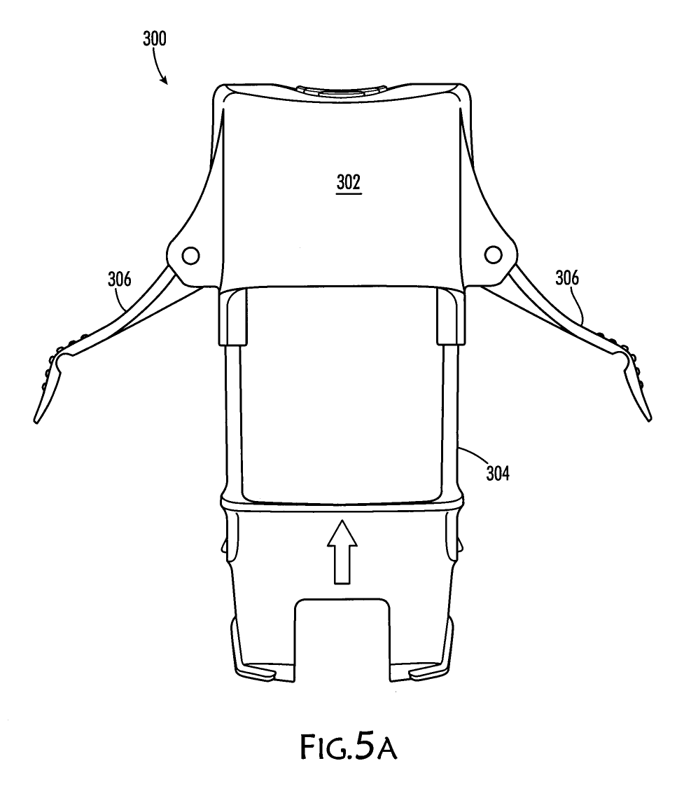

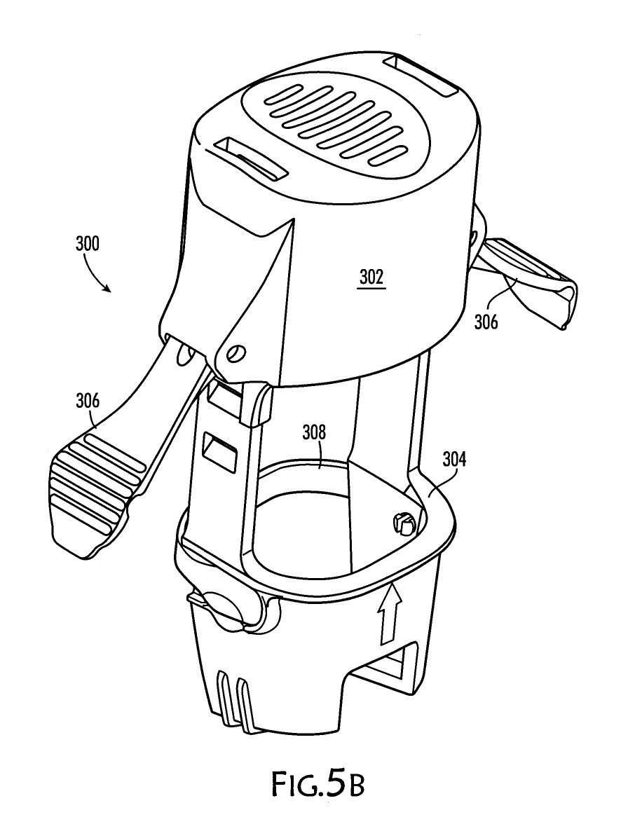

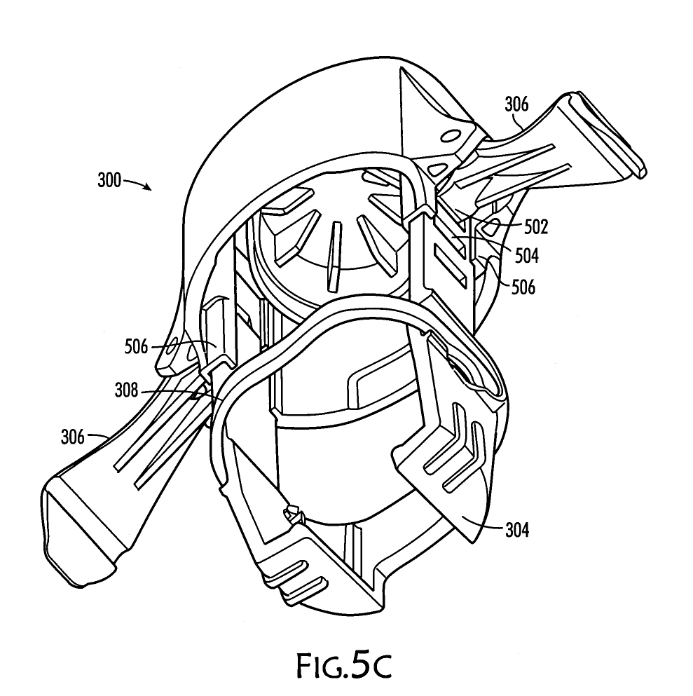

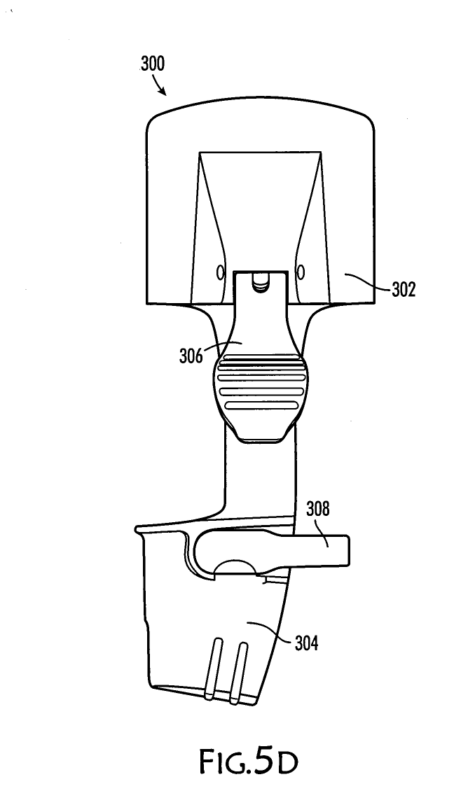

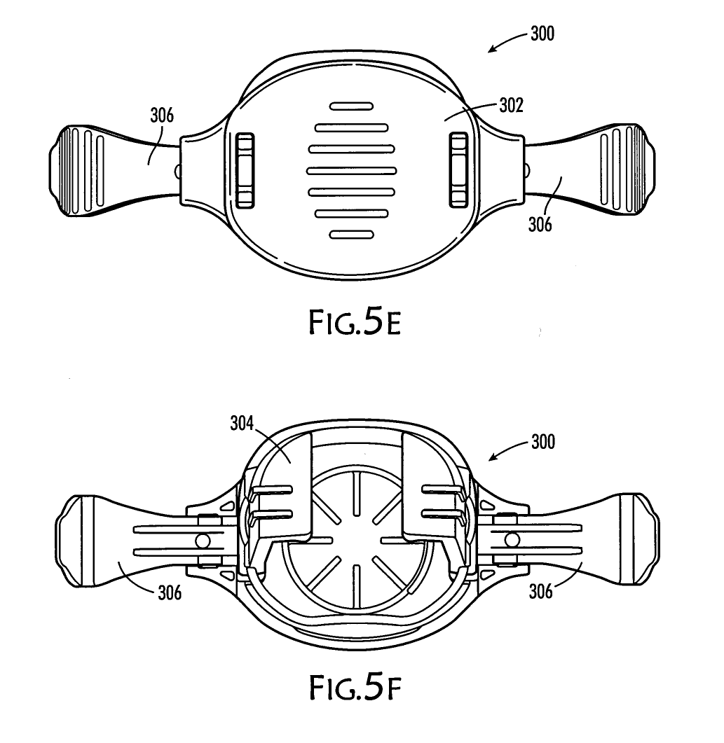

In an aspect, accessory device 300 is configured with a housing 202 including a header 302 and a lower body 304, which together form the housing 202. In an aspect, the lower body 304 is moveable relative to the header 302, the relative movement between the lower body 304 and the header 302 causing activation of the MDI. At least one lever 306 is used as a movement actuator 204 to instigate the movement between the lower body 304 and the header 302, for example with teeth 502 (shown and described in more detail with respect to FIGS. 5C and 6) located on the lever 306 which abut counterpart grooves 504 located on the lower body 304, where rotation of the lever about a pivot point 310 causes the teeth to engage one of the grooves to cause the movement of the lower body 304 and subsequent compression of the aerosol canister 120 as the aerosol canister 120 is pressed against the top of the header 302. In some aspects, two levers 306 are provided to the accessory device 300 to instigate 5 movement and/or activation of the MDI. Optionally, the levers 306 are symmetrical and/or apply symmetrical movement actuation to the housing 202 and/or are located on the side(s) of the housing 202. Optionally, the levers 306 are asymmetrical and/or provide asymmetrical movement actuation. In an aspect, a fastening strap 308 reversibly secures the MDI 100 within the accessory device 300.

It should be understood that in use, the user of the accessory device 300 can use any combination of fingers (including the thumb) to squeeze the side-mounted levers 306 together, in the axis transverse to vertical axis 130, to activate the MDI 100, for example, using the thumb on a first lever and at least one finger on the second lever to squeeze the levers 306. Further, it is conceived that because, in some aspects, the levers 306 are located on the side of the housing 202 providing a substantial “width” dimension, the accessory device 300 including the MDI can rest and/or be retained in and/or by the palm of a patient's hand during activation, enhancing ease of use of the MDI for users that have problems with holding and activating the MDI in a conventional manner.

FIG. 4 is a perspective view of the accessory 300 (without a hand held metered dose inhaler 100), in an aspect. The levers 306 are optionally provided with ergonomic adaptations, such as finger grips 402, to enhance user/patient control. The finger grips 402 are optionally wider and/or thicker than a majority of the lever 306, and/or the finger grips 402 are optionally textured for better gripping effect.

FIGS. 5A-5F are front, perspective back, perspective bottom, side, top and bottom views, respectively, of the accessory 300 of FIGS. 3-4, in an aspect. Accessory 300 is shown in FIG. 5A with the fastening strap 308 removed to provide additional clarity to the inside of the accessory 300 (where the MDI 100 where will be mounted). FIG. 5C shows the accessory form a perspective bottom view where an underside of the top of the header 302 can be seen. In an aspect, the lower body 304 is slidable within a channel 506 of the header 302.

FIG. 6 is an exploded view of the accessory 300 of FIG. 4, showing the header 302 including the channels 506, the lower body 304 including grooves 504, the levers 306 including the teeth 502, the fastening strap 308, and the pivot points 310 where the levers 306 are attached to the header 302.

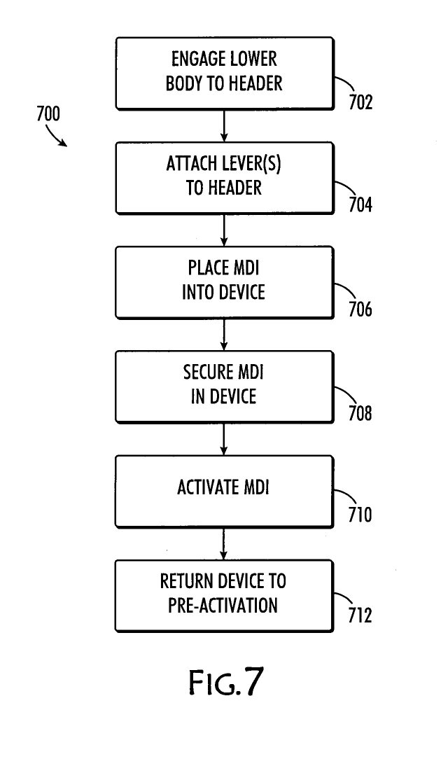

FIG. 7 is a flowchart showing a method 700 of using an accessory device 200, 300 for a MDI 100. As described elsewhere herein, the configurations of accessory devices 200, 300 are designed to make hand held metered dose inhalers easier to use, thereby improving patient compliance and/or enhancing patient medical outcomes and/or ensuring more reliable/consistent patient dosing. In some aspects, the accessory devices 200, 300 are pre-assembled (without the MDI) to make commencement of use easier for the patient, however, in the case that this is not true, the patient would slide the lower body 304 into the header 302 so that they are moveably engaged (702) and/or attach (704) at least one lever 306 at a pivot point 310 onto the header 302, in no particular order. In an aspect, the MDI 100 is placed (706) into the accessory device 300 with the mouth piece 104 extending from the lower body 304 and with the end 122 of the aerosol canister 120 abutting the underside of the top of the header 302. Optionally, the MDI 100 is reversibly secured (708) in the accessory device 300 using a fastener 208, for example the fastening strap 308.

In an aspect, the MDI 100 is activated (710) by squeezing or applying pressure to the at least one lever 306, optionally two levers on opposing sides of the accessory 300 towards each other, whereby the pressure on the lever 306 causes the lower body 304 to move relative to the header 302, compressing the top 122 of the aerosol canister against the header 302 to release a metered dose of the contents of the canister 120. In some aspects, the accessory device 300 is biased to return (712) the lower body 304 to a position relative to the header 302 to the pre-activation (710) configuration after activation (710). Optionally, the accessory device 300 is returned (712) to the pre-activation (710) configuration by the aerosol canister 120 of the MDI 100 decompressing after activation (710).

It is expected that during the life of a patent maturing from this application many relevant accessories for activating a MDI will be developed and the scope of the term “accessory” is intended to include all such new technologies a priori.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures 10 thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Further, described ranges are intended to include numbers outside any range described within statistical error and/or inherent measurement equipment limitations.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. An accessory device for use with a hand-held metered dose inhaler (MDI), comprising:
a housing, sized and shaped to mount the MDI therein;
a movement mechanism configured to cause compressive movement of at least one piece of the housing to activate the MDI; and,
at least one movement actuator, mounted on a side of the housing, operationally connected to the movement mechanism to instigate the movement.

2. An accessory device according to claim 1, further comprising a fastener removably attachable to the housing and at least partially enclosing the MDI to retain the MDI within the housing.

3. An accessory device according to claim 2, wherein the fastener is a fastening strap.

4. An accessory device according to claim 1, wherein the housing comprises a header and a lower body.

5. An accessory device according to claim 4, wherein the lower body is moveably disposed in at least one channel of the header, such that initiation of the movement actuator causes the lower body to move relative to the header by sliding in the channel.

6. An accessory device according to claim 5, wherein the at least one side-mounted movement actuator is mounted on the housing at a pivot point.

7. An accessory device according to claim 5, wherein the at least one side-mounted movement actuator is provided with at least one tooth configured as a counterpart to at least one groove in the header, such that engagement of the groove by the tooth effectuates movement of the lower body relative to the header.

8. An accessory device according to claim 6, wherein the at least one side-mounted movement actuator is a lever.

9. An accessory device according to claim 8, wherein the lever is provided with a textured finger grip.

10. An accessory device according to claim 9, wherein the lever is provided with at least one of a wider portion and thicker portion to enhance user control.

11. A method of using an accessory device with an MDI, comprising:
placing an MDI within a housing of the accessory device;
activating the MDI by causing compressive movement of a movement mechanism by instigating motion of a movement actuator.

12. A method according to claim 11, further comprising sliding a lower body of the accessory device into a header of the accessory device so that they are moveably engaged.

13. A method according to claim 11, further comprising attaching two levers as a movement actuator on opposite sides of the housing at a pivot point.

14. A method according to claim 13, further comprising squeezing the two levers towards each other to cause the activating.

15. A method according to claim 11, further comprising reversibly securing the MDI to the accessory device with a fastener.

16. A method according to claim 12, further comprising sliding the lower body to a position relative to the header to the pre-activation configuration after activation.

17. A method of using an accessory device to transfer activation of an MDI from a vertical axis to an axis transverse to the vertical axis, comprising:
providing a side-mounted movement actuator to a housing of the accessory device, wherein movement of the movement actuator in the transverse axis instigates movement of a movement mechanism which causes compressive movement in a vertical axis of the MDI.

18. A method according to claim 17, wherein the side-mounted movement actuator comprises two opposing levers, movement of which, towards each other, instigates the movement of the movement mechanism.
